SEQUENCE LISTING (2 sequences)

SEQ ID NO: 1 - DNA, 51 bp, Artificial Sequence
  SARS-CoV-2 spike protein binding aptamer
cagcaccgaccttgtgctttgggagtgctggtccaagggcgttaatggaca

SEQ ID NO: 2 - DNA, 26 bp, Artificial Sequence
  VEGF binding aptamer
caattgggcccgtccgtatggtgggt

(19) United States
(12) Patent Application Publication
(10) Pub. No.: US 2024/0218376 A1
(43) Pub. Date: Jul. 4, 2024

(54) APTAMER-TYPE MULTI-WARHEAD COVALENT DRUG

(71) Applicants: Jay YANG, Hokkaido (JP); Yudai TABUCHI, Tokyo (JP); Masumi TAKI, Tokyo (JP)

(72) Inventors: Jay YANG, Hokkaido (JP); Yudai TABUCHI, Tokyo (JP); Masumi TAKI, Tokyo (JP)

(21) Appl. No.: 18/555,113

(22) PCT Filed: Apr. 11, 2022
(86) PCT No.: PCT/JP2022/017487

§ 371 Date: Oct. 12, 2023

(30) Foreign Application Priority Data
Apr. 13, 2021 (JP) 2021-067753

(51) Int. Cl. C12N 15/115; A61P 31/14

(52) U.S. Cl. CPC C12N 15/115; A61P 31/14; C12N 2310/16; C12N 2310/351

(57) ABSTRACT

The invention imparts new properties to nucleic acid aptamers, which may enhance its function as therapeutic drugs or the like. Also provided is a covalent drug having the new properties as an anti-SARS-COV-2 drug. Specifically, an aptamer-based multiwarhead covalent drug having multiple covalent binding warheads is provided. In particular, an aptamer-based multiwarhead covalent drug targeting SARS-COV-2 is provided. A pharmaceutical composition comprising the multiwarhead nucleic acid aptamer and a method of producing the multiwarhead nucleic acid aptamer are also provided.

### CROSS-REFERENCE TO RELATED APPLICATION(S)

This is a national stage application filed under 37 U.S.C. 371 based on International Patent Application No. PCT/JP2022/017487, filed Apr. 11, 2022, which claims priority to Japanese Patent Application No. 2021-067753, filed on Apr. 13, 2021, the disclosures of which are incorporated herein by reference in their entireties.

### SEQUENCE LISTING

The application contains a Sequence Listing which has been submitted electronically in .txt format together with the International Application and is hereby incorporated by reference in its entirety. Said .txt copy, created on Apr. 11, 2022, is named “JPOXMLDOC01-seq1.app.txt” and is 633 bytes in size. The sequence listing contained in this .txt file is part of the specification and is hereby incorporated by reference herein in its entirety.

### TECHNICAL FIELD

The present disclosure relates to compounds and compositions which specifically bind to targets and which are useful as medicaments or as biochemical tools.

### BACKGROUND ART

SARS-COV-2 corona virus is a pathogenic virus causing a pandemic during years 2020-2021. Research for developing drugs to treat the diseases associated with this virus are ongoing world-wide, and further development of therapeutic drugs specific for this virus is still desirable.

Some documents, including Non-Patent Document 1, disclose nucleic acid aptamers that bind to the spike protein of SARS-COV-2. The spike protein is the constituent protein of the protruding structures (spikes) which project from the outer surface of SARS-COV-2 and decorates the viral particle. It is a viral protein which binds to the receptors (e.g., ACE2 receptor or Neuropilin-1 receptor) expressed on the cells of human respiratory systems etc. to mediate infection in humans. The nucleic acid aptamer of Non-Patent Document 1 was identified based on its ability to bind to the receptor binding domain (RBD) of the spike protein and its ability to compete with ACE2 for binding to the RBD.

Non-Patent Documents 2 and 3, which are completely separate lines of studies unrelated to SARS-COV-2, disclose nucleic acid aptamers capable of covalent-binding. Non-Patent Document 2 reports introducing a sulfonyl fluoride (SF) group at the 5′-ends of a nucleic acid library for the in vitro selection method, SELEX, and performing a screening from the library to obtain a nucleic acid aptamer which binds to the epidermal growth factor receptor. Non-Patent Document 3, aiming to address the problem that covalent drugs have the risk of irreversible adverse effects, has introduced a single SF group to a conventional thrombin-binding aptamer and reports a thrombin-inhibiting agent which has a covalent-binding ability and whose drug effect is reversible by a complementary strand antidote.

### PRIOR ART LITERATURE

### Non-Patent Documents

Non-Patent Document 1: Song et al., Anal. Chem., 2020, 92, 9895-9900
Non-Patent Document 2: Yuko SHISHIDO, Hokkaido University Doctoral Thesis, Sep. 25, 2018
Non-Patent Document 3: Tabuchi et al., Chem. Commun., 2021, 57, 2483-2486

### SUMMARY OF THE INVENTION

### Problem to be Solved by the Invention

The embodiments of the present disclosures impart new properties to nucleic acid aptamers which may enhance its usefulness in the functions as therapeutic drugs and the like. The embodiments of the present disclosures also provide covalent-binding agents such as anti-SARS-COV-2 agents having such new properties.

### Solution to the Problem

The present disclosure includes the following embodiments.

[1]

A multiwarhead nucleic acid aptamer having multiple fluorosulfonyl groups, wherein the multiple fluorosulfonyl groups are linked to multiple nucleic acid residues in the nucleic acid sequence of the nucleic acid aptamer via linkers.

[2]

The multiwarhead nucleic acid aptamer according to [1], wherein a first nucleic acid residue linked to a fluorosulfonyl group via a linker and a second nucleic acid residue linked to a fluorosulfonyl group via a linker are at least 3 residues apart.

[3]

The multiwarhead nucleic acid aptamer according to [1] or [2], wherein the multiple fluorosulfonyl groups are linked to the nucleic acid aptamer via an azide-alkyne click chemistry reaction, wherein the linkers include linking moieties formed by the azide-alkyne click chemistry reaction.

[4] The multiwarhead nucleic acid aptamer according to any one of [1]-[3], wherein the fluorosulfonyl groups are linked to multiple nucleic acid residues within a nucleic acid sequence of a SARS-COV-2 spike protein binding nucleic acid aptamer via respective linkers, wherein the multiwarhead nucleic acid aptamer is capable of covalently binding to a SARS-COV-2 spike protein.
[5]

The multiwarhead nucleic acid aptamer according to [4], having the nucleic acid sequence of 5′-CAGCACCGACCTTGTGCTTTGGGAGTGCTGGTCCAAGGGCGTTAATGG ACA-3′ (SEQ ID NO:1), wherein the fluorosulfonyl groups are linked to multiple nucleic acid residues within the nucleic acid sequence via respective linkers.

[6]

The multiwarhead nucleic acid aptamer according to any one of [1]-[5], wherein the fluorosulfonyl groups are present in the form of -aryl-SO₂F.

[7]

A pharmaceutical composition comprising the multiwarhead nucleic acid aptamer of any one of [1]-[6].

[8]

A pharmaceutical composition comprising the multiwarhead nucleic acid aptamer of [4] or [5], for inhibiting the binding of SARS-COV-2 to a receptor on a cell of a subject.

[9] A method of producing a nucleic acid aptamer having an enhanced efficiency for binding to a target protein, the method comprising reacting:
a) a nucleic acid aptamer specific for the target protein, wherein multiple nucleic acid residues within the nucleic acid sequence of the aptamer are each modified to be linked or bound to a first reactive group; and
b) a warhead compound having a structure in which a second reactive group corresponding to the first reactive group is linked or bound to a fluorosulfonyl group to obtain a multiwarhead structure in which the multiple nucleic acid residues of the nucleic acid aptamer are each linked to the fluorosulfonyl group via a linker comprising a linking moiety formed by the reaction between the first reactive group and the second reactive group.
   [10]

The method according to [9], wherein a first nucleic acid residue and a second nucleic acid residue of the multiple nucleic acid residues are at least 3 residues apart.

[11]

The method according to [9] or [10],
wherein the first reactive group is a group having a carbon-carbon triple bond (a1) or an azide group (a2),
wherein the second reactive group corresponding to the first reactive group is an azide group (b1) or a group having a carbon-carbon triple bond (b2), and
wherein the reaction is an azide-alkyne click chemistry reaction.
   [12]

The method according to [9] or [10], wherein the fluorosulfonyl groups in the multiwarhead structure are present in the form of -aryl-SO₂F.

[13]

The method according to [11], wherein the fluorosulfonyl groups in the multiwarhead structure are present in the form of -aryl-SO₂F.

### BRIEF DESCRIPTIONS OF THE FIGURES

FIG. 1 shows a mobility shift assay showing that the SARS-CoV-2 binding nucleic acid aptamers each linked to a warhead at a single site are capable of forming a covalent bond to the target viral protein (RBD) (a), and the results of measuring the amounts of remaining unreacted RBD (b).

FIG. 2 represents an experiment showing that the SARS-CoV-2 binding nucleic acid aptamers, each linked to a warhead at two sites, retain the specific binding ability for the target protein, and demonstrate enhanced efficiency for forming a covalent bond.

FIG. 3 shows the results of investigating the concentration dependency of the covalent binding interactions between the double-warhead aptamers and the target protein.

FIG. 4 represents an experiment showing that the triple-warhead aptamer, linked to the warhead at three sites, also retain the specific binding ability for the target protein and a further enhanced efficiency for forming a covalent bond.

FIG. 5 shows the results of investigating the time course of the covalent binding interactions between the triple-warhead aptamer and the target protein.

FIG. 6 shows the results of investigating the concentration dependency of the covalent binding interactions between the triple-warhead aptamer and the target protein.

FIG. 7 shows the result of an experiment of mixing the target protein and the triple-warhead aptamer in the presence of human serum.

FIG. 8 provides the results of ELISA showing that the aptamer-based multiwarhead covalent drugs strongly inhibit the protein-protein interaction between the SARS-COV-2 viral protein and its human receptor.

FIG. 9 shows that the multiwarhead modified VEGF binding aptamers (T4/T17, T4/T22) have an enhanced efficiency for covalently binding to the VEGF protein compared to the single-warhead modified aptamers (T4, T17, T22).

### DETAILED DESCRIPTION OF THE INVENTION

There is no published precedent on the conception of introducing covalent-binding warheads to multiple sites of a nucleic acid aptamer at the same time. There was no knowledge of whether such introduction of multiple warheads would be feasible at all in a functional drug in the first place, and it was also not known what kind of technical effect such arrangement might bring about. The present inventors have conducted research with this new conception to not only prove that a functional drug can be obtained by linking covalent-binding warheads to multiple sites of a nucleic acid aptamer, but also discover that it can provide novel properties that were not seen in conventional drugs. The present disclosure provides embodiments of aptamer-based multiwarhead covalent drugs based on these discoveries.

Non-Patent Documents 2 and 3 mentioned above have disclosed introducing one covalent-binding warhead (the sulfonyl fluoride group) at one site (and in the case of Non-Patent Document 2, a specific one site which is the 5′-terminus) of the nucleic acid aptamer, but the prior art disclosures have not made the embodiments of the present invention obvious. Firstly, the purpose of the prior arts was to form an irreversible bond between the aptamer drug and the target molecule by the covalent-binding warhead-Non-Patent Document 3 explains that this can increase the “local concentration” of the aptamer at the target molecule. These purposes, i.e. forming an irreversible bond between the drug and the target and increasing the local concentration of the aptamer drug at the location of the target molecule, are in principle completed by a single covalent bond, and there is no obvious reason to increase the number of covalent bonds.

Secondly, the nucleic acid aptamers are drugs that depend on specific molecular arrangements of the single-stranded nucleic acids which are flexible linear structures. There was a possibility that the tensions that could be generated and physical freedom that could be enjoyed within the linear structure might be fundamentally altered when such a flexible linear structure is constrained from multiple separate points, as opposed to when it is constrained at a single point. It was not obvious from the prior arts that a functional aptamer could be formed and maintained in the first place when the nucleic acid aptamer was linked to multiple covalent-bonding warheads at the same time. This was discovered for the first time by the present application. Thirdly, although the fluorosulfonyl group as a covalent-binding warhead was known, attaching it to multiple sites of a drug at the same time lacked precedent, and it was much less predictable what would happen when this was done in an aptamer. The data disclosed in the present application suggest that there is a significant time lag between the aptamer docking and the formation of a covalent bond by the fluorosulfonyl group which is sufficient to permit release of the aptamer, and the presence of the additional fluorosulfonyl groups can significantly decrease this time lag. That knowledge, however, is provided for the first time by the present disclosure. The knowledge that an aptamer which bears multiple fluorosulfonyl groups can avoid becoming a nondiscriminatory binder prone to off-target covalent bonding, but it can rather keep a high target selectivity, is also provided for the first time by the present disclosure.

Fourthly, as shown in the Example section, the covalent drugs according to the embodiments of the present disclosure can covalently bind one molecule of the target efficiently, and depending on the environment, they can also covalently bind multiple molecules of the target. There was previously no covalent drug having a potential to change drug dynamics in this way in response to the environment. This is a novel function that cannot be seen as a mere extension of the prior arts. The present embodiments bring forth the drugs having such new properties into this technical field and are therefore inventive.

The present disclosure provides a multiwarhead nucleic acid aptamer having multiple fluorosulfonyl groups, wherein the multiple fluorosulfonyl groups are linked to multiple nucleic acid residues in the nucleic acid sequence of the nucleic acid aptamer via linkers. The multiwarhead nucleic acid aptamer is capable of, in addition to the specific interaction (docking) to the target protein which is intrinsic to the aptamer, covalently binding the target protein. This compound can be understood as an aptamer-based covalent drug (covalent binding agent). One molecule of the multiwarhead nucleic acid aptamer can form multiple covalent bonds to one target molecule, or depending on the environment, form covalent bonds to multiple target molecules. In the present disclosure, “drug” refers to a substance that can specifically bind to a target substance, particularly a target protein. For example, a drug that can bind to the active site of a target enzyme to inhibit the enzymatic activity is contemplated, as well as a drug which is used simply as a binding label or targeting moiety. Therefore, the term “drug” should be construed as referring to a “binding agent” that can be broadly used in vivo or in vitro.

More specifically, the present disclosure provides a multiwarhead nucleic acid aptamer which has fluorosulfonyl groups linked to multiple nucleic acid residues, i.e., at least two nucleic acid residues, within a nucleic acid sequence of a SARS-COV-2 spike protein binding nucleic acid aptamer via respective linkers, and which is thereby capable of covalently binding to a SARS-COV-2 spike protein. The spike protein binding nucleic acid aptamer is preferably a nucleic acid aptamer which binds to the receptor-binding domain of the spike protein. In one embodiment, provided is a SARS-COV-2 spike protein binding nucleic acid aptamer having the nucleic acid sequence of 5′-CAGCACCGACCTTGTGCTTTGGGAGTGCTGGTCCAAGGGCGTTAATGG ACA-3′ (SEQ ID NO:1), wherein the fluorosulfonyl groups are linked to multiple nucleic acid residues within the nucleic acid sequence via respective linkers. By having such a structure, the compound is capable of specifically recognizing and binding (docking) to the SARS-COV-2 target protein thorough the nucleic acid aptamer moiety represented by SEQ ID NO: 1, and efficiently forming covalent bond(s) to the target through the multiple fluorosulfonyl groups. Below, embodiments of the present disclosure will be described by particularly referring to the examples of SARS-COV-2 spike protein binding nucleic acid aptamers, but it should be appreciated that the descriptions are applicable to other nucleic acid aptamers as well.

### [Nucleic Acid Aptamers]

The term nucleic acid aptamer means a nucleic acid molecule capable of specific binding to a target molecule as understood by a person skilled in the art. The nucleic acid aptamer technology per se is well known to those skilled in the art. Naturally occurring nucleic acid aptamers existing as part of riboswitches are also known, but more typical nucleic acid aptamers in the technological applications are artificial nucleic acid aptamers, and more specifically, nucleic acids selected by screening random sequence libraries for the ability to bind the target substances (the screening is called SELEX (Systematic evolution of ligands by exponential enrichment)), or nucleic acids obtained by partially modifying (e.g., truncating and/or chemically modifying) the nucleic acid sequences thus selected. These are usually non-naturally occurring free nucleic acid fragments comprising (or consisting of) sequences that are not naturally present, or at least, non-naturally occurring free nucleic acid fragments comprising (or consisting of) sequences that do not encounter the target molecules in a natural condition. Since an aptamer is obtained or produced as an entity which binds to a target substance of interest, the specific target is accordingly known for the respective aptamer. Therefore, a person skilled in the art can clearly recognize a nucleic acid aptamer and its corresponding target.

Many aptamers have been published and known to those skilled in the art. Descriptions of many examples of aptamers and known technologies and reagents relating to the aptamers can be found in e.g., aptagen.com, bioanalysis-zone.com, genemedsyn.com, linaris.de, cambio.co.uk, and basepairbio.com. The nucleic acid aptamer having the sequence of SEQ ID NO:1 itself is, as mentioned above, described by Non-Patent Document 1, and it was identified by the authors of Non-Patent Document 1 on the basis of its ability to bind to the receptor binding domain (RBD) of the SARS-COV-2 spike protein and its ability to compete with ACE2 for binding to the RBD. Other examples of SARS-COV-2 spike protein binding aptamers are also described by Non-Patent Document 1. Several other documents also describe nucleic acid aptamers binding to SARS-COV-2 spike protein but having different primary structures. For example, Yang et al., Signal Transduct Target Ther. 2021: 6: 227, Sun et al., Angew. Chem. Int. Ed. 2021, 60, 10266-10272, Valero et al., PNAS 2021 Vol. 118 No. 50 e2112942118, and Schmitz et al., Angew. Chem. Int. Ed. 2021, 60, 10279-10285 describe RNA or DNA aptamers which can inhibit the infection by binding to RBD or non-RBD portions of the SARS-CoV-2 spike protein. These aptamers are essentially similar in that they all function by binding to this protein which decorates the surface of the viral particle at high density and mediates infection to the host cells. What is described and demonstrated for the aptamer of SEQ ID NO: 1 can be observed in other SARS-COV-2 spike protein binding aptamers as well. Although it has been found that the anti-infection effect provided by the conventional SARS-CoV-2 spike protein binding aptamers can be limited despite their high binding affinities manifested by the K_{d} values, the embodiments of the present disclosure can come through with the anti-infection effect through the new means, an aptamer-based multiwarhead covalent drug. Significance of this contribution is large.

Further, the principle of the invention of the present disclosure has been demonstrated for different nucleic acid aptamers, for different targets, and for different combinations of warhead-linkage positions for given nucleic acid aptamers, and therefore it has generality which can be applied to diverse nucleic acid aptamers. Besides the spike protein binding aptamers, specific examples of aptamers which can be converted to multiwarhead covalent drugs according to the present disclosure include, but are not limited to, aptamers binding to thrombin, coagulation Factor IXa, factor Xa, von Willebrand factor, tissue factor pathway inhibitor, platelet-derived growth factor, complement protein C5, nucleolin, CXCL12, CCL2, hepcidin, Toll-like receptor 9, VEGF, and the like. In the Example section, data for multiwarhead VEGF binding aptamers are shown by way of example.

Further nucleic acid sequences may be added to 5′- and/or 3′-side of the nucleic acid aptamer sequence (such as SEQ ID NO: 1). In the SELEX method for identifying nucleic acid aptamers, the nucleic acid members of the nucleic acid library to be screened usually have primer sequences (including the same sequence as the primer and the sequence which is complementary to the primer) at 5′- and 3′-ends for the amplification of the library by a nucleic acid amplification technique such as PCR. In fact, the nucleic acid aptamer of Non-Patent Document 1 was originally isolated/identified as a spike protein binding molecule from a population of nucleic acids which had primer sequences added to both ends and thus had longer sequences. Subsequently, the 5′-end sequence and the 3′-end sequence which were not essential for the specific binding to the target were cut off to shorten the aptamer to the 51mer sequence of SEQ ID NO:1. As in this case, it is also typically seen in other nucleic acid aptamers that different sequences can be added to 5′- and/or 3′-side of the core sequence providing the target-binding ability.

In the present disclosure, a “core sequence” of a nucleic acid aptamer means a nucleic acid sequence from which half or more of the SELEX primer sequence (for example half or more, from the side of the nucleic acid molecular end, of each primer sequence) has been removed, as in SEQ ID NO:1, and which still retains, in isolation, the ability to bind the target.

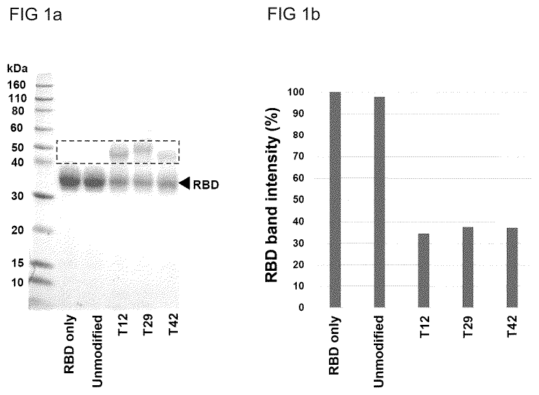

Non-Patent Document 2 describes introducing a sulfonyl fluoride (SF) group at the 5′-ends of a SELEX library for selecting a nucleic acid aptamer for the epidermal growth factor receptor. This is understood to mean the SF group was introduced to the 5′-end of the PCR amplification primer sequence. The present inventors have found that introducing one SF group via a linker to even an internal nucleic acid residue, which is in the middle of the core sequence and away from the primer sequences, does not abolish the specific binding function of the nucleic acid aptamer and allows efficient covalent bond formation (Non-Patent Document 1, FIG. 1 of the present application). Since the primer sequences are present in all members of the SELEX library and the vast majority of the members do not have the target-specific binding ability, it is the core sequence which gives rise to the specific binding ability. It was an unpredicted finding that the function of the nucleic acid aptamer could be retained even when the linker and the SF group were introduced to the core sequence. Some embodiments of the present disclosure are at least partly based on this finding.

A nucleic acid aptamer can specifically bind to the target substance with a high binding affinity expressed by a sub-micromolar (preferably sub 100 nanomolar, more preferably sub-10 nanomolar) dissociation constant K_{d}, and the nucleic acid aptamers of the present embodiments linked to multiple fluorosulfonyl groups may also retain such specific binding ability and affinity for the target substances. Typically, binding between the aptamer and the target is obtained as a sum total of numerous non-covalent binding interactions.

A nucleic acid aptamer may generally be a DNA, an RNA, or a combination thereof, and may be, for example, a single-stranded DNA, a single-stranded RNA, or a combination thereof. Some single-stranded nucleic acid aptamers may partially form self-complementary strands within the molecules. The term nucleic acid aptamer encompasses those comprising one or more nucleic acid modifications or non-natural nucleic acid moieties (such as non-natural backbones and non-natural sugar moieties) known to a person skilled in the art. Various nucleic acid modifications and non-natural nucleic acid moieties which can be used within the aptamers, e.g. for the purpose of nuclease resistance or the like, are known (Zhou et al., Nat Rev Drug Discov, 2017, 16(3): 181-202). Examples thereof include, but are not limited to, 2′-fluorination, 2′-amination, 2′-O-methylation, 3′-modification by an inverted thymidine, phosphorothioate, LNA, and polyethylene glycol (PEG) modifications. The length of a nucleic acid aptamer is generally 10 residues (bases) or longer, more typically 15 residues or longer. The length of a nucleic acid aptamer is typically no longer than 100 residues and more typically no longer than 60 residues, but it can be longer than these. In the present disclosure, a nucleic acid residue (or simply “residue”) means a monomeric unit constituting the nucleic acid polymer. Therefore, for example, each deoxyribonucleotide moiety in a DNA aptamer can be recognized as a residue.

In general, aptamer-type drugs are characterized by the fact that their drug effects can be cancelled, or “antidoted”, by administration of a complementary strand. In Non-Patent Document 3 mentioned above, it was shown that the drug effect of the thrombin covalent-binding aptamer could be cancelled by a complementary strand, but it was also shown that the efficiency of the cancellation became slightly lower compared to the non-covalent-binding aptamer. The present inventors have recognized that cancelling the drug effect has relatively low importance for the SARS-COV-2 binding nucleic acid aptamer which binds not to the human receptor but to the virus. This does not exclude the possibility of cancelling the drug effect of the multiwarhead nucleic acid aptamer by a complementary strand.

The multiwarhead nucleic acid aptamer, or aptamer-based multiwarhead covalent drug, is characterized in that it is linked to fluorosulfonyl groups at a minimum of two nucleic acid residues. The number of the residues linked to fluorosulfonyl groups may be 2, 3, or 4, or it may be even possible to have 5, 6 or more such residues. The number of the residues linked to fluorosulfonyl groups may be for example less than 20% or less than 10% of the residues included in the nucleic acid aptamer. In the present disclosure, the residues linked to the fluorosulfonyl groups correspond to the residues bound to the linkers.

It has been discovered that within the nucleic acid aptamer, the locations of the multiple nucleic acid residues to which the fluorosulfonyl groups are linked may be varied relatively freely. In the studies described in Non-Patent Document 3 and those represented by FIG. 1 of the present application, the present inventors performed experiments using diverse aptamers having different targets, and tried introducing a single warhead at various positions including residues close to the target-binding face and residues far away from the target-binding face, within the aptamer core sequences, but in each case covalent binding to the specific target could be obtained. The present application has taken these studies a step further and discovered that a functional covalent-binding aptamers can be obtained even when two or more of these nucleic acid residues are linked to the warheads at the same time. It has been found that the presence of the multiple warheads could increase the probability of forming a covalent bond at a molecular level compared to the conventional covalent drugs, and improve reaction rates and reaction efficiency. It has also become possible for one molecule of the drug to form covalent bonds to multiple molecules of the target depending on the environment.

At least one, or all, of the residues linked to the fluorosulfonyl groups can be internal residues as opposed to the terminal residues of the nucleic acid aptamer. For example, at least one, or all, of the residues linked to the fluorosulfonyl groups can be residues located at least 5 residues or at least 10 residues internal to the 5′-end or 3′-end of the nucleic acid aptamer. At least one, or all, of the residues linked to the fluorosulfonyl groups can be residues within the core sequence of the nucleic acid aptamer. For example, at least one, or all, of the residues linked to the fluorosulfonyl groups can be residues located at least 5 residues or at least 10 residues internal to the 5′-end or 3′-end of the core sequence of the nucleic acid aptamer.

In a preferable embodiment, at least one, or all, of the linkers are bound to nucleobases in the nucleic acid residues. In another embodiment, the linker may be bound to the 5′-end, or 5′-terminal phosphate (i.e. a phosphate group bound to the 5′-end), of the nucleic acid aptamer. In another embodiment, the linker may be bound to the 3′-end, or 3′-terminal phosphate (i.e. a phosphate group bound to the 3′-end), of the nucleic acid aptamer. In still another embodiment, the linker may be bound to the 2′ position of the ribose ring of any of the residues constituting the nucleic acid aptamer. The number of residues in a nucleic acid aptamer is finite, and the number of sites in each residue to which a linker can be attached is also finite. Therefore, based on the present disclosures and available knowledge, a person skilled in the art can determine a suitable combination of linker attachment sites within a nucleic acid aptamer without undue experimentation and easily verify that the ability to bind the target is not lost.

Nucleic acid residues having alkyne structures or azide groups bound or linked to these locations, which are usable for azide-alkyne click chemistry reactions, as well as modified oligonucleotides of any sequences incorporating such nucleic acid residues, may be commercially available or synthesized by a person skilled in the art. Synthesizing a nucleic acid incorporating multiple such residues can also be done within ordinary skill in the art. Modified-oligonucleotide synthesis services for any sequences, incorporating residues having alkyne structures or azide groups bound or linked to any of the above-described locations, are also commercially available. Examples of the companies providing reagents or custom synthesis service for such alkyne-modified or azide-modified oligonucleotides include, but are not limited to, Integrated DNA Technologies, Inc., Nihon Gene Research Laboratories Inc., and Sigma-Aldrich Co. LLC. These structures have been conventionally used for, e.g. fluorescent labeling of nucleic acids.

When a linker is bound to a nucleobase in the nucleic acid aptamer, the base may be any of adenine (A), guanine (G), inosine (I) (hypoxanthine), cytosine (C), thymine (T), uracil (U), and the like, but A, C, T or U is preferable and T or U is especially preferable. For C and U, for example, the linker may be bound to 5′-position of the pyrimidine ring constituting the base. For T, the linker may be attached by replacing the methyl group at 5′-position of the pyrimidine ring constituting the thymine base, resulting in the same structure as ‘U’ bound to the linker as described above. In the present specification, such a situation may still be described by saying the linker is bound to 5′-position of the pyrimidine ring constituting the T (thymine) base. For A, G, and I, the linker may be bound to 7′-position of the purine ring constituting the base. In such cases, the nitrogen atom at 7′-position of the purine ring may be replaced by a carbon atom.

In an embodiment, a first nucleic acid residue linked to a fluorosulfonyl group via a linker and a second nucleic acid residue linked to a fluorosulfonyl group via a linker are at least 3 residues apart. The first nucleic acid residue and the second nucleic acid residue may be at least 5 residues apart, or at least 10 residues apart. In the present disclosure, a first nucleic acid residue and a second nucleic acid residue being one residue apart means these residues are adjacent to each other, and n residues apart means there are n−1 residues between these residues. The terms “first”, “second” and the like in the present disclosure are used merely for the convenience of referring to distinct elements (e.g., distinct residues). Therefore, the “first nucleic acid residue” and the “second nucleic acid residue” in this paragraph do not refer to any specific positions on the nucleic acid sequence.

In some specific embodiment, a SARS-COV-2-binding nucleic acid aptamer having the sequence of SEQ ID NO: 1 is provided wherein at least one of the multiple positions to which fluorosulfonyl groups are linked correspond to any one of i) to iii) below. In another embodiment, a SARS-COV-2-binding nucleic acid aptamer is provided wherein fluorosulfonyl groups are linked to i) and ii), i) and iii), ii) and iii), or i), ii) and iii) in SEQ ID NO:1 via linkers:
i) a nucleobase of any of A9 to T15;
ii) a nucleobase of any of T26 to T32; and
iii) a nucleobase of any of G39 to A45,
   wherein the alphabets represent the types of bases and the subscript numbers represent the base positions within SEQ ID NO:1.

In a further specific embodiment, a SARS-COV-2-binding nucleic acid aptamer is provided wherein fluorosulfonyl groups are linked to the bases of i) and ii), i) and iii), ii) and iii), or i), ii) and iii) in SEQ ID NO:1 via linkers:
i) T₁₂;
ii) T₂₉; and
iii) T₄₂.

By introducing fluorosulfonyl groups to multiple residues distanced from each other on the nucleic acid sequence as described above, the aptamer's specific docking state will be more robust through the covalent bonding when multiple covalent bonds are formed to a single target, and on the other hand, the probability for the formation of covalent bonds to multiple target molecules may also be increased.

As a side note, the aptamer of Non-Patent Document 1 is a DNA aptamer and therefore originally has T bases. When a linker is bound by replacing the methyl group present in position 5 of the pyrimidine ring of the T base, such a linker-modified T base will have the same structure as a corresponding linker-modified U base. However, since the original aptamer had the sequence of T bases, notations like T₁₂ and T₂₉ are used above. When the same type of linker is bound to a U base of an RNA aptamer, it may be referred to as “a linker bound to a U base” even if the resulting structure is the same.

### [Warhead]

The fluorosulfonyl group may be represented by the formula —SO₂F and is a reactive, electrophilic chemical group known to be capable of reacting with at least a serine, threonine, lysine, tyrosine, cysteine, or histidine residue of a protein under physiological conditions to form a covalent bond (Narayanan et al., Chemical Science 2015, 6(5), 2650-2659). Such a reactive group forming a covalent bond with a target may be referred to as a warhead. In the present disclosure, a nucleic acid aptamer linked to a fluorosulfonyl group may be referred to as a warhead-modified aptamer. In the present disclosure, linkage of multiple fluorosulfonyl groups is referred to by the term “multiwarhead”. As partly illustrated by Narayanan et al. paper, diverse compounds having fluorosulfonyl groups have been synthesized, and diverse synthetic routes are available to a person skilled in the art for introducing a fluorosulfonyl group.

Determining what kind of structure the chemical group of the warhead should be more desirably provided in has been still an immature field. Especially, little knowledge has been accumulated regarding the comparison of behaviors of different warhead structures in the context of aptamer-based covalent drugs, which has made predictions impossible. Other embodiments such as attaching —SO₂F directly to an alkyl linker can be contemplated, but the warhead structures present in the form of -aryl-SO₂F or -heterocyclyl-SO₂F are preferable in the embodiments of the present disclosure because these structures can function especially efficiently in aptamer-based covalent drugs. More specifically, -carbonyl-aryl-SO₂F or -carbonyl-heterocyclyl-SO₂F is preferable, and -carbonyl-aryl-SO₂F such as -carbonyl-phenyl-SO₂F is especially preferable. A heterocyclyl may be, for example, a 1,4-piperadine group. It has been found that these preferable warhead structures may impart to an aptamer a good covalent binding ability regardless of the method of linkage.

### [Linkers]

A person skilled in the art will appreciate that a wide variety of chemical linkers can be used in the present embodiments as long as they can covalently link the nucleic acid aptamer and the fluorosulfonyl group (or the warhead structures discussed above). Chemical linkers typically have hydrocarbon-based backbones. Hydrocarbon-based backbones include those comprising other atoms bound to and/or inserted into the hydrocarbon backbones. The term “linked” as used in the present disclosure means a condition in which two moieties are indirectly bound by having other atom(s) in between, which is distinguished from a condition in which two moieties are directly covalently bound to each other without having other atoms in between. That is, if one moiety (nucleic acid aptamer) is bound to one end of the linker structure, and another moiety (fluorosulfonyl group) is bound to the other end of the linker structure, then the two moieties are “linked”. “Covalently linked” means that two moieties are connected by a series of covalent bond. For example, when one end of the linker is covalently bound to the nucleic acid aptamer and the other end to the fluorosulfonyl group, and the backbone of the linker itself consists of covalent bonds between atoms, then the nucleic acid aptamer and the fluorosulfonyl group are covalently linked.

A person skilled in the art can select and use suitable linkers based on ordinary knowledge and depending on the parameters of the linkers being designed, such as their lengths, hydrophilicity, flexibility, and steric hindrance and convenience of synthesis. For example, the linker may be, but is not limited to, a divalent group comprising or consisting of a C₁₋₁₀ alkylene, C₂₋₁₀ alkenylene, C₂₋₁₀ alkynylene, heteroalkylene, cycloalkylene, heterocycloalkylene, arylene, heteroarylene or other divalent heterocyclic group, —CONH— (or —NHCO—), —CO—, —NH—, —O—, or a combination thereof. Heteroalkylene refers to an alkylene skeleton in which the carbon atom is replaced by one hetero atom per site such as an oxygen, nitrogen, or sulfur atom, and includes ether moieties as well as polyether moieties such as polyethyleneoxy and polypropyleneoxy. Cycloalkylene and heterocycloalkylene refer to alkylene and heteroalkylene that are cyclic, respectively. Heterocycloalkylene includes crown ethers. The groups that make up linkers in the present disclosure include substituted and unsubstituted ones. For example, the hydrogen atoms of alkylene, arylene, heteroarylene, heteroring, and/or —NH— may be substituted. Examples of typical substitution groups include, but are not limited to, C₁₋₁₀ alkyl and alkyloxy, aryl, hydroxyl, amino, halogen, and combinations thereof.

It is especially preferable that at least one, or all, of the linkers in the present embodiments comprise a linking moiety formed by an azide-alkyne click chemistry reaction. Linking moieties formed by other reactive groups and reaction mechanisms (for example, an amide linking moiety formed between an amino group and an N-hydroxysuccinimide (NHS) ester group) may also be feasible, but by using an azide-alkyne click chemistry reaction, linking of the nucleic acid aptamer and multiple fluorosulfonyl groups and production of the multiwarhead covalent drugs can be performed extremely efficiently, such that separation of the product following the reaction may be even omitted. Notably, it has been found that use of the linking moieties formed by azide-alkyne click chemistry reactions may substantially improve covalent binding ability of aptamer-based covalent drugs. Use of these preferable linking moieties may impart to an aptamer a good covalent binding ability regardless of the type of the warhead structure. A possible reason for such a difference in covalent-bonding ability occurring depending on the linking methods could be that the reaction condition for azide-alkyne click chemistry, which can be carried out in a mild solution condition in a short time, can suppress instability or degradation of the warhead compound. Other possibilities could also be considered, for example, the liking structure comprising the nitrogen-rich ring provides a favorable physicochemical effect on the nucleic acid, target protein, and/or —SO₂F group.

Azide-alkyne click chemistry reactions per se are known, and a person skilled in the art can clearly recognize the structure of the linking moiety formed by an azide-alkyne click chemistry reaction. An azide-alkyne click chemistry reaction can be described as a [3+2] cycloaddition reaction occurring between an azide group (—N+N⁺+N⁻) and a carbon-carbon triple bond (—C≡C—) moiety.

One preferable example of the linking moiety formed by an azide-alkyne click chemistry reaction is the divalent group represented by the chemical formula (I) below:

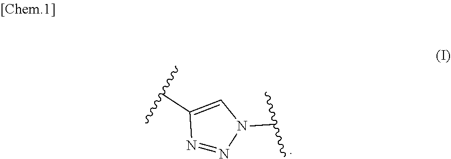 [SMILES: *c1cn(*)nn1]

This is a linking moiety formed by the azide-alkyne click chemistry reaction called copper(I)-catalyzed azide-alkyne cycloaddition (CuAAC). In a preferable embodiment, the left side (left arm) of the above structural formula is linked or bound to the nucleic acid aptamer and the right side (right arm) is linked or bound to the fluorosulfonyl group, but the reverse is also possible.

Another example of a linking moiety formed by an azide-alkyne click chemistry reaction is the divalent group represented by the general formula (II) below. In the general formula (II) below, the 8-membered ring may be substituted by substitution groups (e.g. methyl, methoxy, O═, fluorine atom, or fusion of an aromatic ring or aliphatic ring), and may be for example part of a bicyclo[6.1.0] structure. Further, in the general formula (II) below, the 8-membered ring may be a hetero ring in which one or more carbons not taking part in fusion with another ring are replaced by a non-carbon atom such as nitrogen; Typically, the carbon at the site of attachment to the linker (the left bond arm in the general formula (II) below) may be replaced by nitrogen.

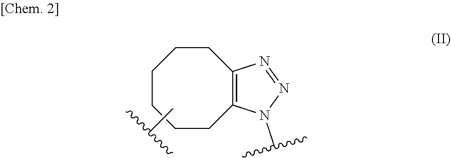

A specific example of the linking moiety represented by the general formula (II) is provided in the chemical formula (IIa) below.

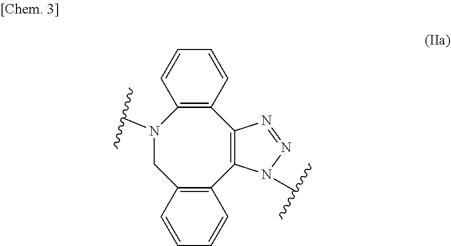

Another specific example of the linking moiety represented by the general formula (II) is provided in the chemical formula (IIb) below.

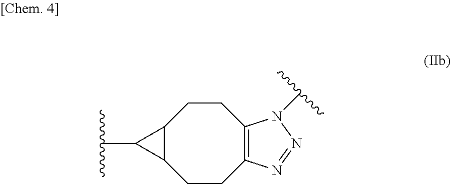

The structures represented by formulas (II), (IIa) and (IIb) are examples of linking moieties formed by azide-alkyne click chemistry reactions called strain-promoted azide-alkyne cycloaddition (SPAAC). SPAAC does not require use of a copper catalyst and therefore is also called a copper-free click chemistry reaction. The left sides (left arms) of the above structural formulas may be linked or bound to the nucleic acid aptamers and the right sides (right arms) to the fluorosulfonyl groups, or vice versa. As described below, the direction of the linking moiety is reversed depending on whether the azide group is provided from the nucleic acid aptamer side or from the fluorosulfonyl group side.

The remaining portions of the linker may be combinations of the groups described above. For example, within the linker, the nucleic acid aptamer side and/or the fluorosulfonyl group side relative to the linking moiety formed by the azide-alkyne click chemistry reaction may be a divalent group comprising or consisting of a C₁₋₁₀ alkylene, C₂₋₁₀ alkenylene, C₂₋₁₀ alkynylene, heteroalkylene, cycloalkylene, heterocycloalkylene, arylene, heteroarylene, —CONH— (or —NHCO—), —CO—, —NH—, —O—, or a combination thereof.

In a specific embodiment, the linker may be represented by formula -L¹-Y-L²-, wherein Y is a linking moiety, e.g., a linking moiety formed by the azide-alkyne click chemistry reaction, L′ is a linker moiety bound to the nucleic acid aptamer (corresponding to the first linker described below), and L² is a linker moiety bound to the fluorosulfonyl group (corresponding to the second linker described below). -L¹-Y-L²- as a whole corresponds to the third linker described below. In one specific example, the third linker consists of the structure —C≡C—(CH₂)₄-(I)-CH₂—CO—C₆H₄—, wherein (I) represents the chemical formula (I).

As described above, the length and specific composition of the linker may be varied depending on the practitioner's design, but just as a guide, the number of carbons in the linker (especially a third linker) may typically be within the range of 2-100, more typically 5-50. Also, just as a guide, the length of the linker, i.e., the distance between the nucleic acid aptamer and the fluorosulfonyl group when the linker is maximally extended may typically be no longer than 100 Å, more typically no longer than 50 Å, or no longer than 30 Å. The length of the linker is typically 5 Å or longer, more typically 10 Å or longer. When the linker is made longer, covalent bonding by the fluorosulfonyl group becomes possible at a site far from the site of the aptamer-binding, or at a site far from the linker-attachment point of the aptamer. The covalent bonds are usually formed within the target molecule(s), but there is a possibility that covalent bonds could be formed to other types of molecules (e.g., other viral proteins) which may be present near the target molecule during the aptamer binding.

### [Pharmaceutical Compositions]

In one aspect, the present disclosure provides pharmaceutical compositions comprising the multiwarhead nucleic acid aptamers described above. In other words, the present disclosure provides the multiwarhead nucleic acid aptamer for use as a medicament. Also provided is the pharmaceutical composition or aptamer for inhibiting binding of SARS-COV-2 to a receptor on a cell of a subject in vitro, ex vivo, or in vivo. The aptamer in this particular case is a multiwarhead-modified spike protein binding aptamer. An embodiment of a method of inhibiting binding of SARS-COV-2 to a receptor on a cell of a subject is also contemplated, the method comprising administering an effective amount of the aptamer, or a composition comprising it, to the subject. A “subject” in the present disclosure is typically a mammal, or tissues or cells thereof. In specific embodiments, a “subject” is a human, or tissues or cells thereof. The subject may be a human patient. The receptor for SARS-COV-2 may be an ACE2 receptor or a Neuropilin-1 receptor.

Inhibiting the binding may include preventing the binding and/or cancelling the binding. It is appreciated that once the receptor binding portion of the spike protein in the virus has been blocked by the covalently fastened nucleic acid aptamer, the virus afterwards will indefinitely reduce or lose the ability to bind to the cells within the host individual, as well as the ability to infect another individual. The SARS-COV-2 binding nucleic acid aptamer or a pharmaceutical composition comprising the same according to the present disclosure may be utilized as a method of prevention of SARS-COV-2 infection, prevention of spread of the infection within an individual of between individuals, or treatment of the infection.

The pharmaceutical composition may further comprise a pharmaceutically acceptable carrier or excipient. A typical example of such a carrier or excipient is water. The pharmaceutical composition may comprise a salt, for example at a concentration suitable for injection into the human body. A person skilled in the art can select suitable administration routes as needed to deliver an effective amount of the pharmaceutical composition to the site or region where the target protein is present. Examples of administration routes include, but are not limited to, oral, intravenous, intra-arterial, intramuscular, subcutaneous, transdermal, intraperitoneal, intrathecal, transrectal, transvaginal, ocular, and by inhalation.

### [Methods of Producing Aptamer-Based Multiwarhead Covalent Drugs]

The methodology and the effects of imparting multiwarhead illustratively demonstrated for the SARS-COV-2 binding nucleic acid aptamer are applicable for other nucleic acid aptamers as well. Therefore, the present disclosure more generally provides methods of producing aptamer-based multiwarhead covalent drugs, and the aptamer-based multiwarhead covalent drugs produced by the methods.

It should be appreciated that this method of production can produce the described aptamer-based multiwarhead covalent drugs which are exemplified by the SARS-COV-2 binding nucleic acid aptamer. Therefore, the structural and functional descriptions provided in the preceding sections in relation to the nucleic acid aptamer aspect herein can be applied to the production method aspect, and vice versa. In the aspect of the production method, a person skilled in the art can select any combinations of target proteins and nucleic acid aptamers specific thereto from known ones not limited to SARS-COV-2. The method of this aspect may be described also as a method of imparting a new covalent-binding functionality to a nucleic acid aptamer, or a method of enhancing the binding efficiency of a covalent binding nucleic acid aptamer.

The present disclosure in one aspect provides a method of producing a nucleic acid aptamer having an enhanced efficiency for binding to a target protein, the method comprising reacting:
a) a nucleic acid aptamer specific for the target protein, wherein multiple nucleic acid residues within the nucleic acid sequence of the aptamer are each modified to be linked or bound to a first reactive group; and
b) a warhead compound having a structure in which a second reactive group corresponding to the first reactive group is linked or bound to a fluorosulfonyl group
to obtain a multiwarhead structure in which the multiple nucleic acid residues of the nucleic acid aptamer are each linked to the fluorosulfonyl group via a linker comprising a linking moiety formed by the reaction between the first reactive group and the second reactive group.

The enhanced efficiency for binding to the target protein means that the aptamer is imparted with an ability of covalent binding compared to a nucleic acid aptamer having the same sequence but not having an ability of covalent binding, or, the aptamer has one or more of the following compared to a nucleic acid aptamer having the same sequence but having only one warhead under the same condition: an increased rate of covalent bond formation; an ability to form a covalent bond at a lower concentration to the target; an ability to form covalent bonds to more (i.e. multiple) target molecules; or an ability to more strongly inhibit the binding of a different molecule which competes for the target protein.

Within the nucleic acid sequence of the aptamer, the number of residues having the first reactive group may be 2, 3, or 4, or it may be even possible to have 5, 6 or more such residues. The number of residues having the first reactive group may be for example less than 20% or less than 10% of the total number of residues included in the nucleic acid aptamer. In the present disclosure, the residues having the first reactive groups correspond to the residues bound to the linkers linking the fluorosulfonyl groups.

Of the multiple nucleic acid residues having the first reactive groups, a first nucleic acid residue and a second nucleic acid residue are preferably at least 3 residues apart. A first nucleic acid residue and a second nucleic acid residue may be at least 5 residues or at least 10 residues apart.

At least one, or all, of the nucleic acid residues having the first reactive groups may be internal residues as opposed to the terminal residues of the nucleic acid aptamer. For example, at least one, or all, of the nucleic acid residues having the first reactive groups may be residues located at least 5 residues or at least 10 residues internal to the 5′-end or 3′-end of the nucleic acid aptamer. At least one, or all, of the residues having the first reactive groups may be residues within the core sequence of the nucleic acid aptamer. For example, at least one, or all, of the nucleic acid residues having the first reactive groups may be residues located at least 5 residues or at least 10 residues internal to the 5′-end or 3′-end of the core sequence of the nucleic acid aptamer.

In a preferable embodiment, at least one or all of the multiple first reactive groups are linked or bound to nucleobases in the nucleic acid residues. In another embodiment, the first reactive group may be linked or bound to the 5′-end, or 5′-terminal phosphate (i.e. a phosphate group bound to the 5′-end), of the nucleic acid aptamer. In another embodiment, the first reactive group may be linked or bound to the 3′-end, or 3′-terminal phosphate (i.e. a phosphate group bound to the 3′-end), of the nucleic acid aptamer. In still another embodiment, the first reactive group may be bound to the 2′ position of the ribose ring of any of the residues constituting the nucleic acid aptamer. The number of residues in a nucleic acid aptamer is finite, and the number of sites in each residue to which a linker can be attached is also finite. Therefore, based on the present disclosures and available knowledge, a person skilled in the art can determine a suitable combination of linker attachment sites without undue experimentation and easily verify that the ability to bind the target is not lost.

The second reactive group of (b) “corresponding to” the first reactive group of (a) means that the combination of the first reactive group and the second reactive group is in a relationship which allows a linking reaction known to those skilled in the art between the two groups. For example, it is especially preferable that the first reactive group of (a) be a group having a carbon-carbon triple bond (—C≡C—) (a1) or an azide group (a2), and the second reactive group of (b) corresponding to the first reactive group be an azide group (b1) or a group having a carbon-carbon triple bond (—C≡C—) (b2), wherein the reaction is an azide-alkyne click chemistry reaction. That is, if the group of (a) is an alkyne structure group (a1), then the group of (b) will be an azide group (b1), for example, and conversely, if the group of (a) is an azide group (a2), then the group of (b) will be an alkyne structure group (b2). It is also possible to form a linking moiety by other reactive groups and reaction mechanisms known to those skilled in the art. For instance in a different example, the first reactive group of (a) is an NHS ester group (a3) or an amino group (a4), and the second reactive group of (b) is an amino group (b3) or an NHS ester group (b4).

The linking reaction in the method of the present embodiments is a reaction to produce a multiwarhead type aptamer, and as such it is understood to involve multiple first reactive groups and corresponding multiple second reactive groups. The term “first reactive group” is used merely for the convenience of referring to a reactive group present on the nucleic acid aptamer side as opposed to the warhead compound side, and the multiple first reactive groups are not necessarily required to have exactly the same structures as each other.

In the present disclosure, a group having a carbon-carbon triple bond usable for an azide-alkyne click chemistry reaction, including alkynyl, cycloalkynyl, or heterocycloalkynyl group, may be referred to as an “alkyne structure”.

The alkynyl group may be based on a terminal alkyne or an internal alkyne. Cycloalkynyl and heterocycloalkynyl groups usable for azide-alkyne click chemistry reactions are known to a person skilled in the art, usually 7-membered or 8-membered, and may be substituted with substitution groups (e.g., methyl, methoxy, O═, fluorine atom, or fusion to an aromatic or aliphatic ring). The heterocycloalkynyl group is a cycloalkynyl group in which one or more carbons in its ring are replaced by a non-carbon atom such as nitrogen. Typically, a carbon at the site in the ring which is bound to the linker may be replaced by nitrogen. The cycloalkynyl and heterocycloalkynyl groups may be used for SPAAC described above, thus they may undergo a click chemistry reaction in the absence of copper (I) ion. The azide group may be expressed by the formula —N₃.

Examples of the alkyne structures known to those skilled in the art as usable for SPAAC include monovalent groups derived from OCT (cyclooctyne), MOFO (monofluorinated cyclooctyne), DIFO (difluorinated cyclooctyne), DIMAC (dimethoxyazacyclooctyne), DIFBO (difluorobenzocyclooctyne), DIBO (dibenzocyclooctyne), DIBAC (dibenzoazacyclooctyne), BARAC (biarylazacyclooctynone), and BCN (bicyclo[6.1.0]nonyne), respectively.

A specific example of the heterocycloalkynyl group is shown in chemical formula III and a specific example of cycloalkynyl group is shown in chemical formula IV below.

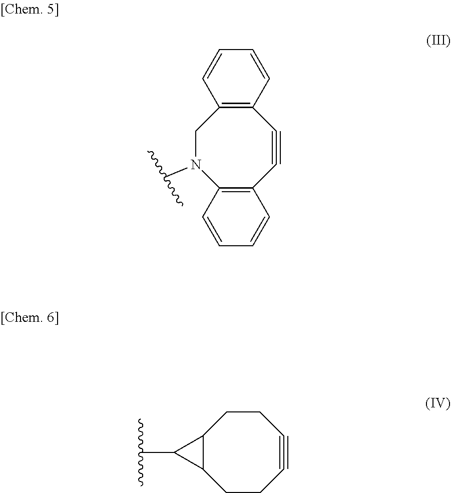 [SMILES: *N1Cc2ccccc2C#Cc2ccccc21 ; *C1C2CCC#CCCC12]

The first reactive group may be directly bound to the nucleic acid aptamer, or linked to the nucleic acid aptamer via a linker. In particular, cycloalkynyl groups, heterocycloalkynyl groups, and azide groups are typically linked to the nucleic acid aptamer via a linker. In the present disclosure, the linker linking the nucleic acid aptamer and the first reactive group may be called a first linker.

A person skilled in the art can select, and use, a suitable first linker according to the parameters of the linker being designed, such as its length, hydrophilicity, flexibility and steric hindrance, as well as convenience of synthesis. For example, the first linker may be, but is not limited to, a divalent group comprising or consisting of C₁₋₁₀ alkylene, C₂₋₁₀ alkenylene, C₂₋₁₀ alkynylene, heteroalkylene, cycloalkylene, heterocycloalkylene, arylene, heteroarylene, —CONH— (or —NHCO—), —CO—, —NH—, —O—, or any combination thereof. The above-listed groups constituting a linker may include substituted and unsubstituted ones. For example, a hydrogen atom of an alkylene, arylene, heteroarylene, and/or —NH— may be substituted. Examples of typical substitution groups include, but are not limited to, C₁₋₁₀ alkyl and alkyloxy, aryl, hydroxyl, amino, halogen, and combinations thereof.

Non-limiting examples of the first linker linking the nucleic acid aptamer (left) to the alkynyl group (right) are recited below: —(CH₂)ₙ— (where n is an integer between 1 and 10); —O—CH₂—; —(CH₂)ₙ—NHCO—(CH₂)₂—O—CH₂— (where n is an integer between 1 and 10); —C≡C—(CH₂)ₙ— (where n is an integer between 1 and 10); and —C≡C—(CH₂CH₂O)ₙ—(CH₂)₂— (where n is an integer between 1 and 10). A specific example of the first linker is —C≡C—(CH₂)₄—.

Non-limiting examples of the first linker linking the nucleic acid aptamer (left) to the cycloalkynyl or heterocycloalkynyl group (right) are recited below: —(CH₂CH₂O)ₙ—(CH₂)₂—NHCO—(CH₂)₄—(CO)— (where n is an integer between 1 and 10); —(CH₂)ₙ—NHCO—(CH₂)₄—(CO)— (where n is an integer between 1 and 10); —(CH₂)ₙ— (where n is an integer between 1 and 10); —(CH₂)₂—O—(CH₂)₂—NHCOO—CH₂—; —(CH₂)ₙ—NHCOO—CH₂— (where n is an integer between 1 and 10); —(CH₂)₆—NHCO—(CH₂)₃—CONH—(CH₂CH₂O)₂—(CH₂)₂—NHCOO—CH₂—.

The term warhead compound refers to one of the two compounds involved in the linking reactions or the present embodiments, and means the compound having the fluorosulfonyl group which provides the covalent-bonding ability of the covalent drug. The second reactive group of the warhead compound may be directly bound to the fluorosulfonyl group or linked to the fluorosulfonyl group via a linker. In the present disclosure, the linker linking the fluorosulfonyl group and the second reactive group may be called a second linker.

A person skilled in the art can select, and use, a suitable second linker according to the parameters of the linker being designed, such as its length, hydrophilicity, flexibility and steric hindrance, as well as convenience of synthesis. For example, the second linker may be, but is not limited to, a divalent group comprising or consisting of C₁₋₁₀ alkylene, C₂₋₁₀ alkenylene, C₂₋₁₀ alkynylene, heteroalkylene, cycloalkylene, heterocycloalkylene, arylene, heteroarylene, —CONH— (or —NHCO—), —CO—, —NH—, —O—, or any combination thereof. The above-listed groups constituting a linker include substituted and unsubstituted ones. For example, a hydrogen atom of an alkylene, arylene, heteroarylene, and/or —NH— may be substituted. Examples of typical substitution groups include, but are not limited to, C₁₋₁₀ alkyl and alkyloxy, aryl, hydroxyl, amino, halogen, and combinations thereof. In particular, the end portion of the second linker bound to the fluorosulfonyl group is preferably an arylene (e.g. p-phenylene) or a heterocycloalkylene (e.g. 1,4-piperazinylene), and more preferably carbonyl-arylene or carbonyl-heterocycloalkylene. These groups constituting the linker may include substituted and unsubstituted ones. For example, a hydrogen atom of an alkylene, arylene, heteroarylene, and/or —NH— may be substituted. Examples of typical substitution groups include, but are not limited to, C₁₋₁₀ alkyl and alkyloxy, aryl, hydroxyl, amino, halogen, and combinations thereof. A specific example of the second linker is —CH₂—CO—C₆H₄—.

Some specific but non-limiting examples of the warhead compounds are shown in chemical formulas (V)-(X) below. In each formula, the moiety linking the azide group and the fluorosulfonyl group is the second linker. The compounds shown below all have an azide group, but the warhead compounds having other second reactive groups described above (e.g., a group having a carbon-carbon triple bond) instead of the azide group are also contemplated. In each of the chemical formulas, the monovalent group which includes from the carbonyl group to the fluorosulfonyl group represents an example of a preferable warhead structure.

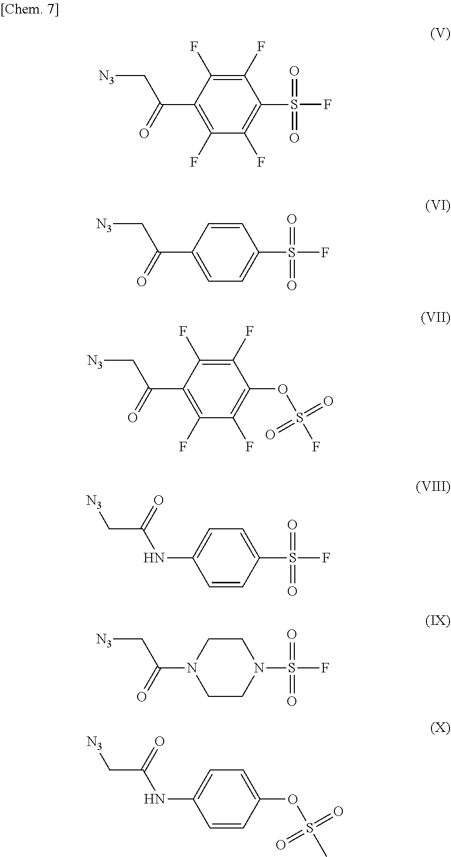

Specific reaction conditions for azide-alkyne click chemistry reactions are well known to those skilled in the art. For example, an azide-alkyne click chemistry reaction can be carried out by mixing an alkyne-structure-containing compound and an azide-group-containing compound in an aqueous solution in the presence of a copper(I)-stabilizing ligand such as Tris(3-hydroxypropyltriazolylmethyl)amine and a reducing agent such as ascorbic acid, and copper(II) sulfate. The copper catalysis system is not necessary for SPAAC. Azide-alkyne click chemistry reactions can be carried out at room temperature.

By a linking reaction known to those skilled in the art (especially an azide-alkyne click chemistry reaction), the above-described linking moiety derived from the first and second reactive groups is formed. As a result, a multiwarhead structure in which multiple residues of the nucleic acid aptamer and the multiple fluorosulfonyl groups are linked via the linker comprising this linking moiety (which is called “third linker” in the present disclosure) is created. This structure can provide an embodiment of the multiwarhead-modified nucleic acid aptamer as well as an active component of the covalent drug composition. The third linker may be a divalent group having the structure: -(first linker)-(said linking moiety)-(second linker)-.

The following Scheme 1 shows a specific example of a synthesis of a warhead compound and a linking reaction between the warhead compound and a nucleic acid aptamer modified by a first reactive group (alkynyl group) via a linker at the nucleobase. Shown are a warhead compound precursor (1), the warhead compound (2), an internal residue of the alkynyl linker-modified nucleic acid aptamer (3), and the internal residue of the warhead-modified nucleic acid aptamer (4).

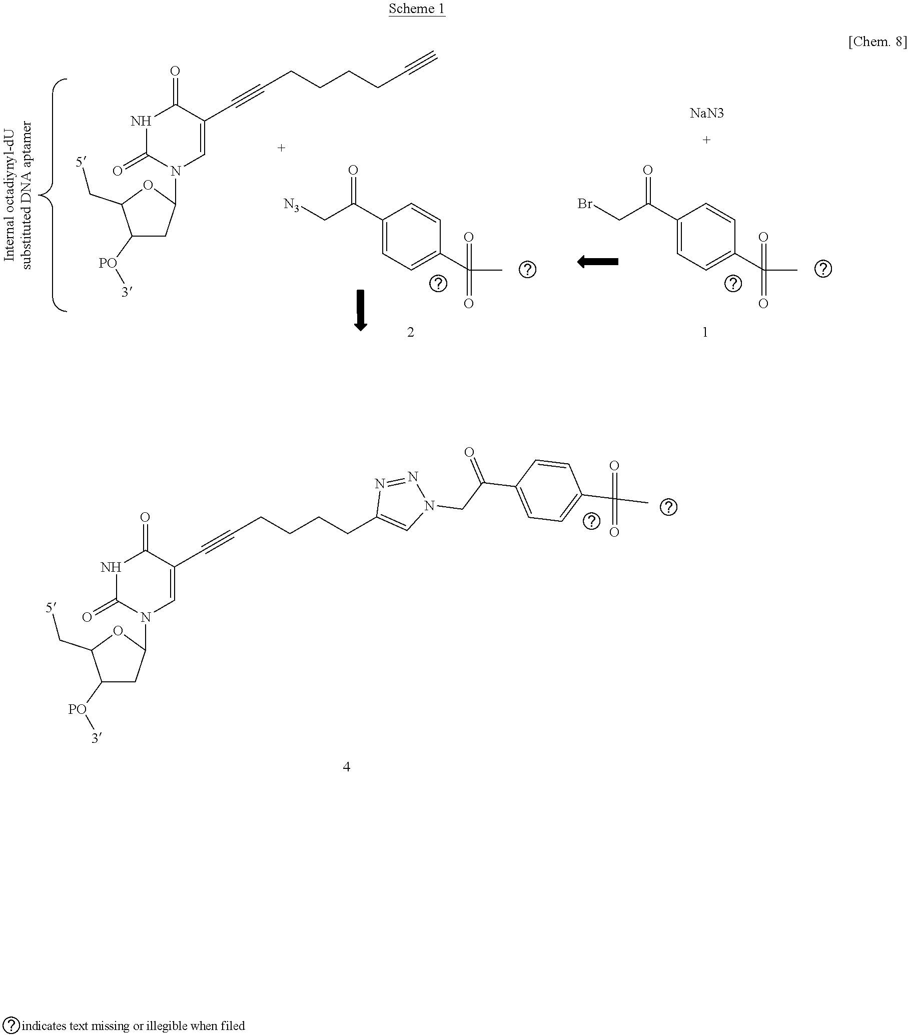

The present method may further comprise chemically synthesizing the nucleic acid aptamer of (a), prior to performing the linking reaction described above. The nucleic acid aptamer components of (a) in the present embodiments are, essentially, modified oligonucleotides, and as such can be synthesized by applying known oligonucleotide chemical synthesis methods. The phosphoramidite method is one example of a suitable chemical synthesis method, but examples are not necessarily limited to it.

For example, a nucleic acid aptamer linked or bound to the first reactive groups at multiple residues may be chemically synthesized, by chemically synthesizing a modified oligonucleotide using at least one nucleoside (e.g. in the form of a phosphoramidite derivative) having a nucleobase to which the first reactive group is linked or bound, as a substrate for the desired multiple residues in the desired sequence.

Nucleic acid aptamers can have high drug efficacy as well as safety due to their properties, including the ability to provide high target-specificity, the ability to be affected by the antidote, and the fact that they scarcely cause immune reactions by themselves, and therefore nucleic acid aptamers are considered promising pharmaceutical agents. On the other hand, the low stability in vivo, or more specifically the short half-life in the circulation because of removal by the kidney, is considered a shortcoming of the aptamer-based pharmaceutical agents and this is one reason why progress of their clinical applications has been slow. The aptamer-based covalent drug provided by the embodiments of the present disclosure will bind to the target protein, and then, once its warhead portion has formed a covalent bond against the target protein, the drug will be retained on or near the target protein, to be able to maintain the effect on the target protein. Therefore, the aptamer-based covalent drug can for example achieve a sustained drug effect in a pharmacokinetics different from that of a conventional aptamer-based drug having a passive and short half-life. The aptamer-based multiwarhead covalent drugs according to the present disclosure can not only significantly improve these advantages, but also provide a disparate effect not seen in conventional aptamer-based drugs or conventional covalent drugs, as shown in the following Examples.

### Examples

Below, examples will be shown to describe embodiments of the present disclosure in greater detail. However, these examples are merely for illustrations, and the present invention is not limited to these specific embodiments illustrated.

### Materials and Methods

### 1. General

An unmodified SARS-COV-2 spike protein-binding aptamer comprising the sequence 5′-CAGCACCGACCT₁₂TGTGCTTTGGGAGTGCT₂₉GGTCCAAGGGCGT₄₂TAATG GACA′ (SEQ ID NO:1) described in Non-Patent Document 1, and alkyne-linker-modified aptamers each comprising the above sequence in which one, two or three sites among T at position 12 (T₁₂), T at position 29 (T₂₉), and T at position 42 (T₄₂) have been replaced with an alkyne-linker-modified T, were custom synthesized at Integrated DNA Technologies, Inc. These alkyne-linker-modified Ts have —C≡C—(CH₂)₄—C≡CH, instead of the usual methyl group, bound to position 5 of the pyrimidine ring of the thymine (T) base. Here, —C≡C—(CH₂)₄— can be recognized as a first linker and —C≡CH as an alkynyl group.

The receptor binding domain of the SARS-COV-2 spike protein (#SPD-C52H3) was purchased from ACROBiosystems, U.S.A. Below, this receptor binding domain of the SARS-COV-2 spike protein is abbreviated as RBD. Human serum (#H4522) was purchased from Aldrich, U.S.A.

NMR experiments were performed by using a 500 MHz nuclear magnetic resonance device (JNM-ECA500, JEOL Ltd.) at 25° C. High performance liquid chromatography (HPLC) analyses were performed on an Agilent 1100 HPLC system (Agilent Technologies, Inc., U.S.A.) connected to a photodiode array (PDA) and/or LCQ-Fleet ion trap mass spectrometer and equipped with a C18 reversed-phase column (Hypersil GOLD, 2.1×100 mm, Thermo Fisher Scientific Inc., U.S.A.) using a 0-100% acetonitrile gradient comprising 0.1% formic acid at a 300 μL/min flow rate. A small scale quantitative analysis of the aptamers was carried out by using a reversed-phase semi-micro HPLC system (PU-2085 with C18 column, JASCO Corporation) connected to a fluorescence detector followed by a PDA. The aptamers were separated using a 0-60% acetonitrile gradient comprising a 20 mM triethylamine acetate aqueous solution (pH 7.4) for 26 minutes at a flow rate of 200 μL/min.

All images of stained gels and in-gel fluorescence were captured by ChemDoc XRS+ (BIO-RAD Laboratories Inc., U.S.A.) and band intensities were quantified by using Image Lab™ software (BIO-RAD Laboratories Inc., U.S.A.).

### 2. Synthesis of Covalent-Binding Aptamers

### 2.1. Synthesis of Warhead Compound 1

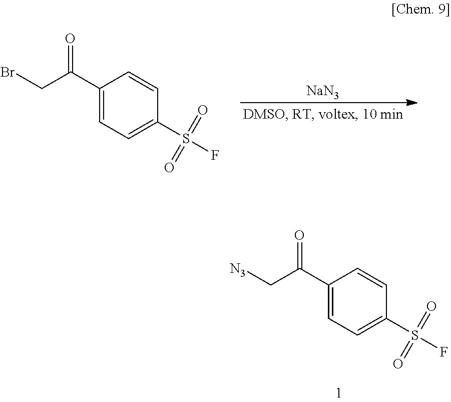 [SMILES: O=C(CBr)c1ccc(S(=O)(=O)F)cc1 ; [N-]=[N+]=NCC(=O)c1ccc(S(=O)(=O)F)cc1 ; "NaN3"]

The warhead compound 1 was synthesized on a preparative scale according to the following procedure.

4-(2-bromoacetyl)-benzene-1-sulfonyl fluoride (71.1 μmol, Aldrich, U.S.A., #00364) and sodium azide (64.6 μmol, Wako Pure Chemical Industries, Ltd., #195-11092) were mixed in 323 μL of dimethyl sulfoxide (DMSO). The reaction mixture was vortexed for 10 minutes at room temperature, then mixed with cold water (0.5 mL) and extracted with ethyl acetate (1 mL). The collected organic phase was washed with saturated NaHCO₃ (0.5 mL×2) and brine (0.5 mL×2), dried over Na₂SO₄, and evaporated to afford a pure product as a yellow solid (11.8 mg, yield 49%). The warhead compound 1 shown on the right side of the figure above was identified by 1H and ¹³C NMRs. In this compound, the acetyl-benzene moiety can be recognized as a second linker

### 2.2. Synthesis of Aptamers Linked to Warheads

Tris(3-hydroxypropyltriazolylmethyl (0.5 μmol in water, Aldrich, U.S.A., #762342) and copper(II) sulfate (0.25 μmol in water, Aldrich, U.S.A., #451657) were mixed. Then, each alkyne-linker-modified aptamer (10 nmol in water) and 50 mM warhead compound 1 (0.5 μmol in DMSO) were added, and following further addition of ascorbic acid (0.4 μmol in water, Aldrich, U.S.A., #A92902), the mixture was reacted for 1 hour at room temperature. The reaction product was purified through ethanol precipitation. Specifically, the crude reaction product was combined with 3M sodium acetate (9 μmol in water) and ethanol, which had been cooled to −20° C., and incubated at −20° ° C. for 1 hour. Then centrifugation was carried out (15000 rpm, 20 minutes, 4° C.), supernatant was removed, and the precipitate was washed with a 70% ethanol solution and dissolved in nuclease-free water. The pure aptamers each linked to the warhead were identified by HPLC analysis.

Below, the SARS-COV-2 spike protein-binding aptamer is abbreviated as SC2BA, and the warhead-modified aptamers in particular are abbreviated as T12, T12/T29, etc. In these abbreviations, T represents the type of the base modified with the warhead (T=thymine), and the numbers such as 12 and 29 represent the positions of the warhead-modified residues in SEQ ID NO:1. If multiple residues are indicated with slash (“/”) separating them, it means these multiple residues are warhead-modified.

### 3. Covalent Bond Mobility-Shift Assay

The unmodified SC2BA and the SC2BAs to which one or more warheads had been linked (60 μM each) were each mixed with RBD (6 μM) in phosphate-buffered saline (D-PBS, pH 7.4) and incubated at 37ºC for 12 hours. The mixture was then combined with 1× sample buffer containing β-Mercaptoethanol, denatured in boiling water, and then separated by 12% sodium dodecyl sulfate poly-acrylamide gel electrophoresis (SDS-PAGE). Total proteins on the gel were visualized by Coomassie brilliant blue (CBB) staining. When SC2BA was covalently bonded to RBD, it was detectable as a mobility shift of the RBD on the gel.

The bands of unreacted RBD (i.e., RBD that had not undergone a covalent-bonding reaction with the aptamer) were quantified by Image Lab™ software. Intensity of the unreacted RBD band in the sample with no addition of the modified SC2BA was normalized to 100%, and relative intensity of that band in each of the other samples was determined.

The foregoing describes basic experimental conditions. As indicated in the figures, reagent concentrations and reaction times were modified as appropriate depending on the purpose of each experiment.

### 4. ELISA (Enzyme-Linked Immunosorbent Assay)

The COVID-19 Spike-ACE2 binding assay kit II from RayBio was used. This assay uses a plate having the human ACE2 receptor protein immobilized on the surface thereof to quantify ACE2-RBD binding, and the assay involves adding a Fc-tagged RBD protein to the plate to cause ACE2-RBD interaction, followed by washes, then reacting an HRP-labeled anti-Fc antibody and measuring color development of the substrate (TMB: 3,3′,5,5′-tetramethylbenzidine) caused by the HRP enzyme. In the experiments to investigate the effect of the aptamer administration, the Fc-labeled RBD protein was first reacted with the unmodified SC2BA or the SC2BAs linked to one or more warheads (100 μM). This reaction solution was then added to the ACE2-immobilized plate to cause protein-protein interaction, followed by washing. Finally, the HRP-labeled anti-Fc antibody was reacted and color development of the substrate for the HRP enzyme was measured to quantify how much the presence of the aptamers could inhibit the ACE2-RBD binding. In connection with this methodology, it was also confirmed that similar inhibition activity could be seen when the human ACE2 receptor protein and the Fc-labeled RBD protein were allowed to interact first and then each SC2BA was added.

### Results and Discussion

The outline of the reaction for linking the fluorosulfonyl group (warhead) to the nucleic acid aptamer is as shown in Scheme 1 above. In this example, the fluorosulfonyl group is linked not to a sequence terminus of the aptamer but to an internal base of the sequence. Moreover, although the fluorosulfonyl group is linked to a thymine base in this example, it is not necessarily limited to a thymine, and the fluorosulfonyl group may be linked to other positions using a similar approach. Although not shown in a figure, the result of HPLC analysis of the reaction mixture following the reaction without any subsequent purification exhibited the molecule in which introduction of the warhead had been completed, at a high purity of at least 90% or greater, regardless of the positions and the number of the linkers.

FIG. 1 (a) shows a result of visualizing the proteins by CBB after incubating the unmodified SC2BA or the warhead-modified SC2BAs with RBD under a physiologically active condition of 37° C. for 12 hours and then separating the reaction mixtures by SDS-PAGE. The 1st lane of the gel shown contains molecular weight markers. RBD by itself has an apparent molecular weight of about 32 kDa (2nd lane). Even if this was bound by the unmodified SC2BA, mobility change of RBD does not occur when run in an SDS-PAGE gel having a reducing/denaturing condition (3rd lane). In contrast, when the warhead-modified SC2BA linked to a fluorosulfonyl group via a linker at position 12, position 29, or position 42 of the nucleic acid sequence (T12, T29, and T42, respectively) was bound, the aptamer formed a covalent bond to RBD, and thus a mobility shift of RBD on SDS-PAGE, i.e. an increase in the molecular weight, was confirmed (4^{th}-6^{th} lanes, dashed box). Correspondingly, the amount of the band that was not mobility shifting, i.e. RBD not covalently bound, was reduced. Quantitative measurement of the intensity of this covalently unbound RBD band revealed that covalent bonds were formed to about 60-65% of RBD in the system (b). A simulation by Non-Patent Document 1 has estimated that position 29 and position 42 of SC2BA are located nearly on the binding face for the RBD, while position 12 is distanced from the RBD binding face.

The same experiment as above was performed in the presence of BSA (bovine serum albumin) in an amount comparable to that of RBD, but the rate of formation of a covalent bond to RBD was substantially the same as the above experiment, and BSA mobility shift was not detected (not shown in the figure). This demonstrates the target specificity of the covalent binding aptamers. Further, the same experiment as above was performed with twice the concentration of the covalent binding aptamers. In this case, the rate of formation of a covalent bond to RBD was increased by a few percents, but the rate did not exceed 70% (not shown in the figure).

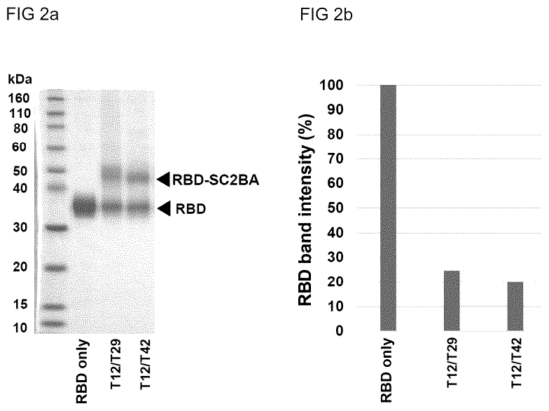

Next, double-warhead aptamers were produced, in which two residues per molecule, namely T12/T29 or T12/T42, were linked to fluorosulfonyl groups, and the same mobility shift assay as above was performed. Surprisingly, introduction of linkers at two positions of the nucleic acid aptamer main body and linkage of two warheads did not result in a loss of the target specific binding ability of these aptamers. On the contrary, significant improvement in the binding efficiency, with covalent bonding rates exceeding 70%, was achieved (FIG. 2).

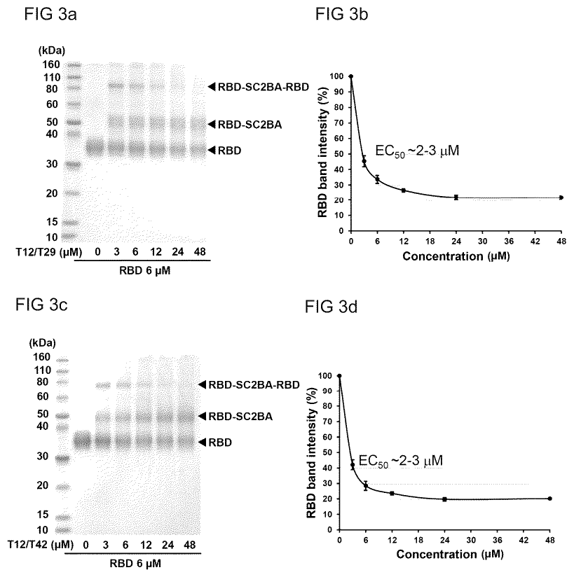

FIG. 3 shows the results of investigating the concentration dependency of covalent binding interactions between the double-warhead aptamers and the target protein. FIG. 3 and b show the results for T12/T29 and c and d show the results for T12/T42. EC₅₀ was determined to be about 2 to 3 μM in each case. It was found that, in addition to the bimolecular covalent binding complex of target protein-aptamer (RBD-SC2BA), a trimolecular covalent binding complex of target protein-aptamer-target protein (RBD-SC2BA-RBD) may also be generated in a concentration-dependent manner. This will be discussed below. In a time course study of the covalent binding interactions between the double-warhead aptamers and the target protein, Tin was determined to be about 30 minutes in each case, indicating that most of the covalent binding reactions occurs rapidly within one hour (not shown in the figure).

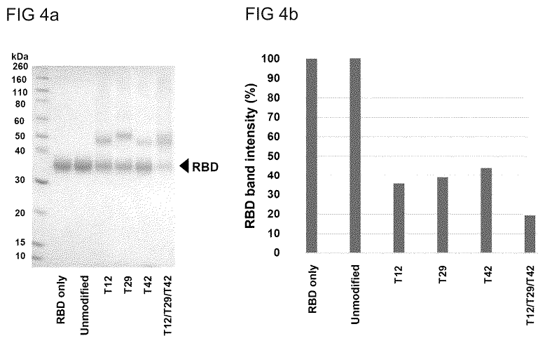

In view of the surprising finding that introduction of linkers and warheads at two different positions in the nucleic acid aptamer core sequence did not result in a loss of the target specific interaction of the aptamers but, on the contrary, resulted in significant improvement of the covalent binding efficiency, a triple-warhead was produced and experiments conducted, in which the linkers and the warheads were introduced to all three of positions 12, 29, and 42. Contrary to the prediction that the increase of the linkers/warheads would have a structurally adverse influence, the target-specific binding ability of the aptamer was still robustly retained, and a significantly high covalent bonding rate that exceeded 80% was achieved (FIG. 4).

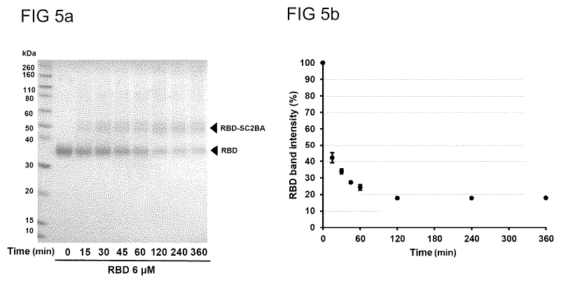

FIG. 5 shows the results of investigating the time course of covalent binding interaction between the triple-warhead aptamer (24 μM) and the target protein (6 μM). Within 15 minutes of mixing the aptamer and the target protein, covalent bonds were formed to over 50% of the target proteins, with the reaction almost complete within 60 minutes. This is a rather fast reaction rate for covalent drugs.

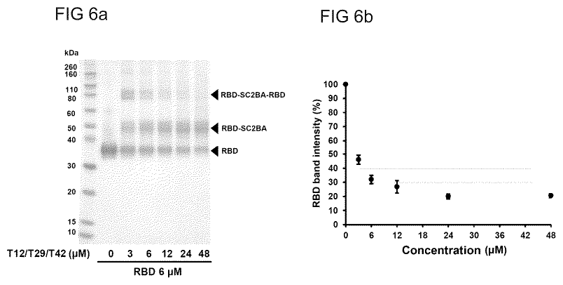

FIG. 6 (a) shows the results of investigating the concentration dependency of covalent binding interactions between the triple-warhead aptamer and the target protein. When the concentration of the aptamer against that of the target protein (RBD) was relatively low, one molecule of the aptamer was found to be able to form covalent bonds to two molecules of the target protein (RBD-SC2BA-RBD). This had been also seen in the experiments using the aptamers having two warheads (FIG. 3). Non-Patent Document 3 was also suggesting that after formation of one covalent bond, the docking between the aptamer moiety and the target molecule could be temporarily dissociated to create a moment in which the two parts are tethered to each other by the linker. In the case of a multi-warhead aptamer, it is believed that the said moment can create a chance for the aptamer to undergo a new docking and covalent bonding with another molecule of the target. This phenomenon is unique to the aptamer-based multiwarhead covalent drugs and represents a novel drug mechanism not known in conventional covalent drugs. As a result of this mechanism, it is seen that more than 50% of the target proteins could form covalent bonds even when the aptamer administered is present only at 50% of the concentration of the target proteins (FIGS. 3b and 3d, FIG. 6 b).

As conspicuously seen in the RBD-SC2BA-RBD bands in FIG. 6 (a), the covalent binding complexes sometimes appear as split bands on the gel in these experiments. In the case of multiwarhead aptamers, there are in theory a plurality of possible combinations on the basis of different warheads making covalent bonds to different positions on different target molecules, and it appears that such plurality of complexed molecular species are actually giving slightly different electrophoretic mobility rates.

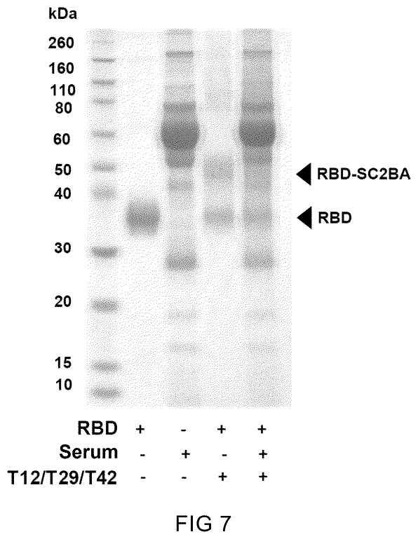

FIG. 7 shows the result of an experiment of mixing the target protein RBD and the triple-warhead aptamer in the presence of human serum. Despite the presence of the high concentration of serum proteins, the extent of covalent binding to the target protein was not substantially reduced (see the intensity of the “RBD” band). A possibility of small amount of non-specific reactions to the serum proteins cannot be excluded, but considering the mass ratios of the proteins, it is clear that the vast majority of the covalent binding reactions had occurred selectively to RBD.

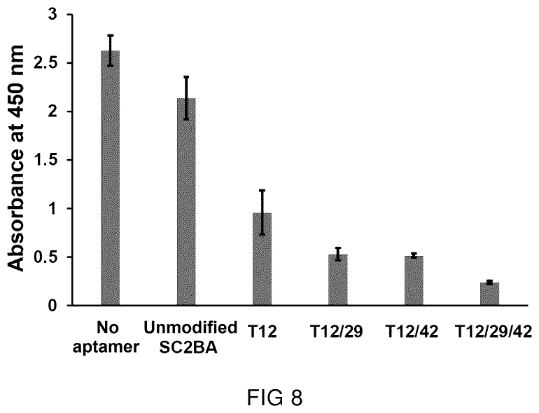

The human ACE2 is a major receptor for SARS-COV-2 and it is an entry point for SARS-COV-2 infecting a human. FIG. 8 shows the results of the ELISA experiment, which assays protein-protein interactions between the RBD of SARS-CoV-2 and the human ACE2, and aims to investigate extent to which the administered aptamer drug can inhibit the interactions between the virus and the human receptor. As has been demonstrated by the studies using antibodies, inhibition of the RBD-ACE2 interaction results in inhibition of infection of the cells by the SARS-COV-2 virus.

It has been reported that K_{d} (dissociation constant) of ACE2 against RBD is 34.6 nM, and K_{d} of the unmodified SC2BA against RBD is about 5.8 nM (Non-Patent Document 1). The results of FIG. 8 show that the unmodified SC2BA indeed has an ability to inhibit the interaction between SARS-COV-2 RBD and human ACE2 but its effect could be limited. However, the aptamer imparted with a covalent binding ability, by introduction of one warhead, significantly improved the efficacy of inhibiting the interaction of the viral protein and the human receptor (the example of T12 is shown in the figure). Moreover, the aptamers incorporating multiple covalent binding warheads exhibited inhibitory capacity enhanced to a surprisingly high level. In the case of the triple-warhead aptamer, the interaction of the viral protein and the human receptor was inhibited at least 90% or even more (FIG. 8).

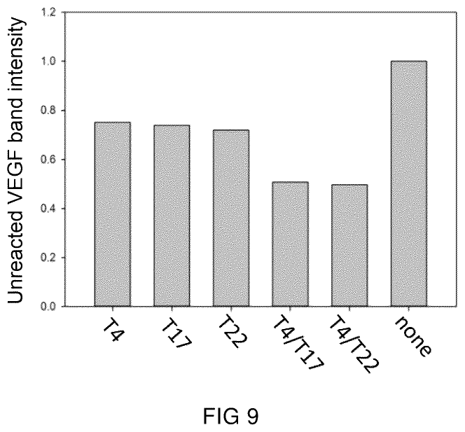

As emphasized above, the principle disclosed herein can be also applied to other nucleic acid aptamers, and what has been described and demonstrated for the SARS-COV-2 spike protein binding aptamer can be observed in other nucleic acid aptamers as well. FIG. 9 as one such example shows data for the vascular endothelial growth factor (VEGF) binding aptamer. This VEGF binding aptamer (5′-CAATTGGGCCCGTCCGTATGGTGGGT-3′; SEQ ID NO:2) per se has been described by Kaur and Yung, PLOS ONE, 2021, 7:e31196. VEGF is a potent angiogenic mitogen commonly overexpressed in cancerous cells. This nucleic acid aptamer was produced by Kaur et al. by truncating an existing, longer VEGF binding aptamer, and has an extremely high target-binding affinity with a K_{d} value of 0.5±0.32 nM. Multiwarhead-modified VEGF binding aptamers were produced essentially as described above, reacted to a fixed amount of recombinant VEGF165 protein (VES-H6248, ACROBiosystems), and intensity of the unreacted (i.e. not forming a covalent bond) VEGF band was quantified on SDS-PAGE. In the experiments shown in FIG. 9, the concentration of the aptamer in each sample is 100 μM. An accurate molecular weight and molar concentration could not be determined because of the glycosylation of VEGF, but at any rate, this was a condition providing the aptamer in excess of VEGF. “none” represents the sample of VEGF protein by itself to which no aptamer has been added. This was used as a reference point of an unreacted band for relative quantification. The multiwarhead-modified VEGF-binding aptamers (T4/T17 and T4/T22; the numbers indicate the residue numbers in SEQ ID NO:2) showed clear enhancement of the efficiency of covalent binding to the VEGF protein compared to the single-warhead modified aptamers (T4, T17, and T22).

The results illustrated in these Examples demonstrate that linking multiple covalent binding warheads to a nucleic acid aptamer generally does not result in a loss of the structural integrity and the target-specific binding ability of the aptamer, and on the contrary, it can significantly enhance the pharmacological effect which is based on the specific binding, by making the covalent bond formation to the target faster, and sometimes enabling covalent bond formation to multiple molecules of the target. Particularly, in these Examples, the aptamer-based multiwarhead covalent drug was demonstrated as a specific and a potentially effective drug against SARS-COV-2.

As shown in the Examples, the aptamer-based multiwarhead covalent drug can present a new aspect as an Environment Responsive (ER) covalent drug. That is, when the drug concentration is relatively low, each drug molecule can fight a lone battle covalently binding two target molecules per one drug molecule, whereas when the drug concentration is relatively high, each single drug molecule can make a concentrated attack on one target molecule to form multiple covalent bonds to it. To the knowledge of the present inventors, such a covalent drug, capable of changing the covalent binding property in this way depending on the environment, has not been previously reported.

1. A multiwarhead nucleic acid aptamer having multiple fluorosulfonyl groups, wherein the multiple fluorosulfonyl groups are linked to multiple nucleic acid residues in the nucleic acid sequence of the nucleic acid aptamer via linkers.

2. The multiwarhead nucleic acid aptamer according to claim 1, wherein a first nucleic acid residue linked to a fluorosulfonyl group via a linker and a second nucleic acid residue linked to a fluorosulfonyl group via a linker are at least 3 residues apart.

3. The multiwarhead nucleic acid aptamer according to claim 1, wherein the multiple fluorosulfonyl groups are linked to the nucleic acid aptamer via an azide-alkyne click chemistry reaction, wherein the linkers include linking moieties formed by the azide-alkyne click chemistry reaction.

4. The multiwarhead nucleic acid aptamer according to claim 1, wherein the fluorosulfonyl groups are linked to multiple nucleic acid residues within a nucleic acid sequence of a SARS-COV-2 spike protein binding nucleic acid aptamer via respective linkers, wherein the multiwarhead nucleic acid aptamer is capable of covalently binding to a SARS-CoV-2 spike protein.

5. The multiwarhead nucleic acid aptamer according to claim 4, having the nucleic acid sequence of 5′-CAGCACCGACCTTGTGCTTTGGGAGTGCTGGTCCAAGGGCGTTAATGGACA-3′ (SEQ ID NO:1), wherein the fluorosulfonyl groups are linked to multiple nucleic acid residues within the nucleic acid sequence via respective linkers.

6. The multiwarhead nucleic acid aptamer according to claim 1, wherein the fluorosulfonyl groups are present in the form of -aryl-SO₂F.

7. A pharmaceutical composition comprising the multiwarhead nucleic acid aptamer of claim 1.

8. A pharmaceutical composition comprising the multiwarhead nucleic acid aptamer of claim 4, for inhibiting the binding of SARS-COV-2 to a receptor on a cell of a subject.

9. A method of producing a nucleic acid aptamer having an enhanced efficiency for binding to a target protein, the method comprising reacting:
a) a nucleic acid aptamer specific for the target protein, wherein multiple nucleic acid residues within the nucleic acid sequence of the aptamer are each modified to be linked or bound to a first reactive group; and
b) a warhead compound having a structure in which a second reactive group corresponding to the first reactive group is linked or bound to a fluorosulfonyl group
to obtain a multiwarhead structure in which the multiple nucleic acid residues of the nucleic acid aptamer are each linked to the fluorosulfonyl group via a linker comprising a linking moiety formed by the reaction between the first reactive group and the second reactive group.

10. The method according to claim 9, wherein a first nucleic acid residue and a second nucleic acid residue of the multiple nucleic acid residues are at least 3 residues apart.

11. The method according to claim 9,
wherein the first reactive group is a group having a carbon-carbon triple bond (a1) or an azide group (a2),
wherein the second reactive group corresponding to the first reactive group is an azide group (b1) or a group having a carbon-carbon triple bond (b2), and
wherein the reaction is an azide-alkyne click chemistry reaction.

12. The method according to claim 9, wherein the fluorosulfonyl groups in the multiwarhead structure are present in the form of -aryl-SO₂F.

13. The method according to claim 11, wherein the fluorosulfonyl groups in the multiwarhead structure are present in the form of -aryl-SO₂F.

14. The multiwarhead nucleic acid aptamer according to claim 3, wherein the fluorosulfonyl groups are present in the form of -aryl-SO₂F.

15. A pharmaceutical composition comprising the multiwarhead nucleic acid aptamer of claim 3.

16. A pharmaceutical composition comprising the multiwarhead nucleic acid aptamer of claim 5, for inhibiting the binding of SARS-COV-2 to a receptor on a cell of a subject.
